(19) United States
(12) Patent Application Publication
(10) Pub. No.: US 2008/0097512 A1
(43) Pub. Date: Apr. 24, 2008

(54) Complex Medical Balloons

(76) Inventors: Scott B. Sidwell, Hollywood, FL (US); Thomas N. Trotta, Miami Shores, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/667,367

(22) PCT Filed: Nov. 18, 2005
(86) PCT No.: PCT/US05/42241

§ 371 Date: May 9, 2007

Related U.S. Application Data
(60) Provisional application No. 60/629,211, filed on Nov. 18, 2004.

(51) Int. Cl. A61M 29/00

(52) U.S. Cl. 606/192

(57) ABSTRACT

A complex medical balloon (12) has at least two sections (14, 16), each of which has a different compliance curve. A compliance curve measures expansion size at various pressures. Accordingly, at a given pressure, one balloon section of a complex balloon will experience greater expansion than another section.

### CROSS-REFERENCE TO RELATED APPLICATION

This application claims priority of U.S. Provisional Patent Application No. 60/629,211, filed Nov. 18, 2004.

### BACKGROUND AND SUMMARY OF THE INVENTION

1. Technical Background

The present invention relates generally to medical devices, and more particularly to catheters having complex balloons.

2. Discussion

Balloon catheters are used in a variety of therapeutic applications, including intravascular catheters for procedures such as angioplasty and/or deploying medical devices such as stents. Approximately one million angioplasties are performed worldwide each year to treat vascular disease, including coronary, peripheral and neurological blood vessels partially or totally blocked or narrowed by a lesion, stenosis, thrombosis, and/or vasospasm.

By way of example, the present invention will be described in relation to angioplasty treatments and stenting. However, it should be understood that the present invention relates to any catheter with a complex balloon having components of differing polymers bonded together, according to the present invention as recited in the following claims, and it is not limited to angioplasty, or stents, or even use in blood vessels.

Most balloon catheters have a relatively long and flexible tubular shaft defining one or more passages or lumens, and have an inflatable balloon attached near one end of the shaft. This end of the catheter where the balloon is located is customarily referred to as the “distal” end, while the other end is called the “proximal” end. The proximal end of the shaft is generally coupled to a hub, which defines a proximal inflation port and may define a proximal guidewire port. If the proximal guidewire port is defined at the hub, the resulting arrangement is referred to as “over-the-wire.” The proximal inflation port communicates with an inflation lumen defined by the shaft, which extends and is connected to the interior of the balloon, for the purpose of selectively inflating and deflating the balloon.

The proximal guidewire port communicates with a guidewire lumen defined by the shaft, for slidingly receiving a guidewire. The guidewire lumen extends between the proximal guidewire port, and a distal guidewire port at the distal end of the catheter. If the proximal guidewire port is located at some intermediate point along the shaft, the resulting configuration is called “rapid-exchange.”

In general, complex balloons according to the present invention have more than one segment, and one segment is more compliant than the other segment. In other words, as the balloon is inflated, each segment will expand at different rates. The complex medical balloon thus exhibits a compound compliance curve.

Various possible structures may be used. For example, the balloon itself may define an inflatable central portion defining an inflated size, flanked by a pair of proximal and distal conical portions, flanked by a pair of proximal and distal legs or collars. The proximal and distal collars may be affixed to a catheter shaft.

This disclosure of the present invention will include various possible features and embodiments. However, the present invention scope as set forth in each of the claims, and is not limited to the particular arrangements described in this disclosure.

Common treatment methods for using such a balloon catheter include advancing a guidewire into the body of a patient, by directing the guidewire distal end percutaneously through an incision and along a body passage until it is located within or beyond the desired site. The term “desired site” refers to the location in the patient's body currently selected for treatment by a health care professional. The guidewire may be advanced before, or simultaneously with, a balloon catheter. When the guidewire is within the balloon catheter guidewire lumen, the balloon catheter may be advanced or withdrawn along a path defined by the guidewire. After the balloon is disposed within the desired site, it can be selectively inflated to press outward on the body passage at relatively high pressure to a relatively constant diameter, in the case of an inelastic or non-compliant balloon material.

This outward pressing of a constriction or narrowing at the desired site in a body passage is intended to partially or completely re-open or dilate that body passageway or lumen, increasing its inner diameter or cross-sectional area. In the case of a blood vessel, this procedure is referred to as angioplasty. The objective of this procedure is to increase the inner diameter or cross-sectional area of the vessel passage or lumen through which blood flows, to encourage greater blood flow through the newly expanded vessel. The narrowing of the body passageway lumen is called a lesion or stenosis, and may be formed of hard plaque or viscous thrombus.

Some balloon catheters are used to deliver and deploy stents or other medical devices, in a manner generally known in the art. Stents, for example, are generally tubular scaffolds for holding a vessel or body passage open. Among other things, complex medical balloon may be used to more accurately stent body passages or vessels at a desired site which includes a branch, or bifurcation.

These and various other objects, advantages and features of the invention will become apparent from the following description and claims, when considered in conjunction with the appended drawings.

### BRIEF DESCRIPTION OF THE DRAWINGS

FIG. 1 is an external perspective view of an over-the-wire balloon catheter;

FIG. 2 is a side elevation view of a complex medical balloon;

FIG. 3 is a diagram of a compound compliance curve;

FIGS. 4-6 are side elevation views of complex medical balloons;

FIGS. 7-8 are side elevation views of a balloon catheter with a complex medical balloon, delivering and deploying a second stent in a cross-section of a side branch passage;

FIGS. 9 and 10 are side elevation views of a balloon and a stent, deflated and inflated, respectively; and

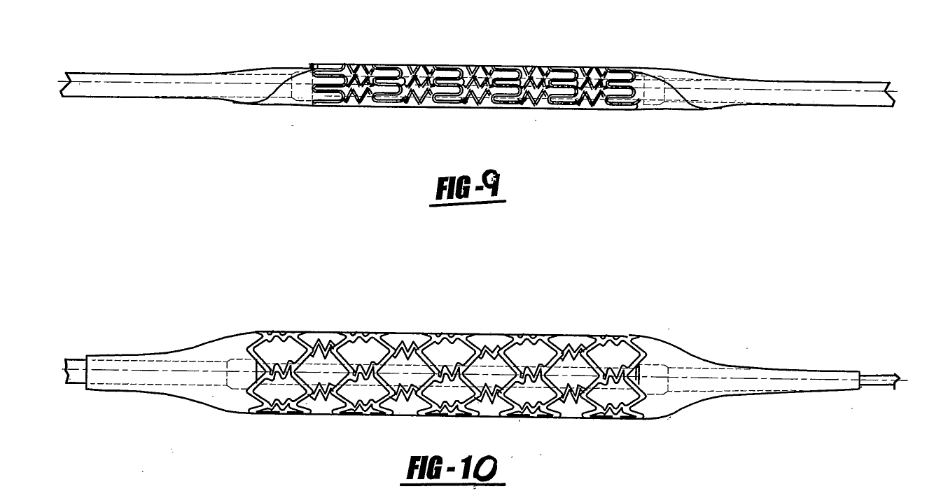

FIG. 11 is an external perspective view of a rapid-exchange balloon catheter.

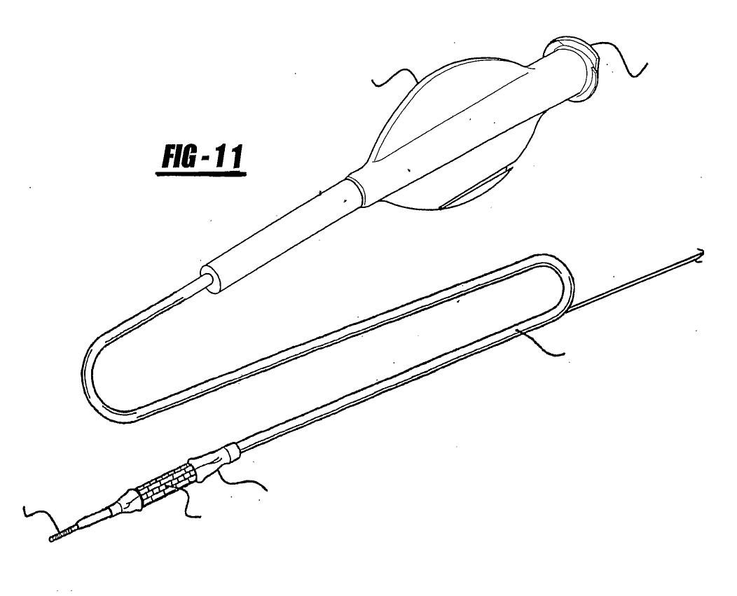

### DETAILED DESCRIPTION OF THE PREFERRED EMBODIMENT

The following description of the preferred embodiments of the present invention is merely illustrative in nature, and as such it does not limit in any way the present invention, its application, or uses. Numerous modifications may be made by those skilled in the art without departing from the true spirit and scope of the invention.

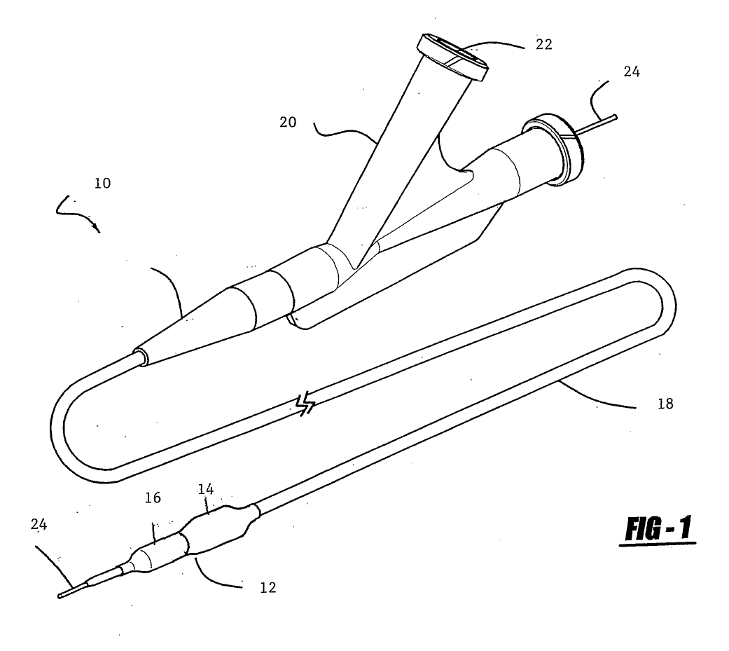

Referring to the drawings, a balloon catheter is depicted at reference number 10 in FIG. 1. The balloon catheter 10 has an complex medical balloon 12 having a proximal and distal balloon segment 14 and 16, a relatively long and flexible tubular shaft 18, and a hub 20. The balloon 12 is affixed to the shaft 18 near a distal end of the shaft 18, and the hub 20 is affixed to the proximal end of the shaft 18.

The shaft defines at least two passages or lumens, one of which is an inflation lumen connected to the balloon 12 for selectively inflating and deflating the balloon 12. The inflation lumen thus provides fluid communication between an interior of the balloon 12, and a hub inflation port 22 having a coupling or luer-lock fitting at the proximal end for connecting the inflation lumen to a source of pressurized inflation fluid (not shown) in the conventional manner.

A second lumen defined by the catheter 10 is a guidewire lumen adapted to receive an elongated flexible guidewire 24 in a sliding fashion. The guidewire 24 and catheter 10 may thus be advanced or withdrawn independently, or the catheter 10 may be guided along a path selected with the guidewire 24.

The balloon 12 has a first and second portion, each defining an inflated size and a working length, flanked by a pair of tapering conical segments, flanked by a pair of “legs” or collars. The proximal collar is affixed to the shaft, and the distal collar is affixed to the shaft near its distal end.

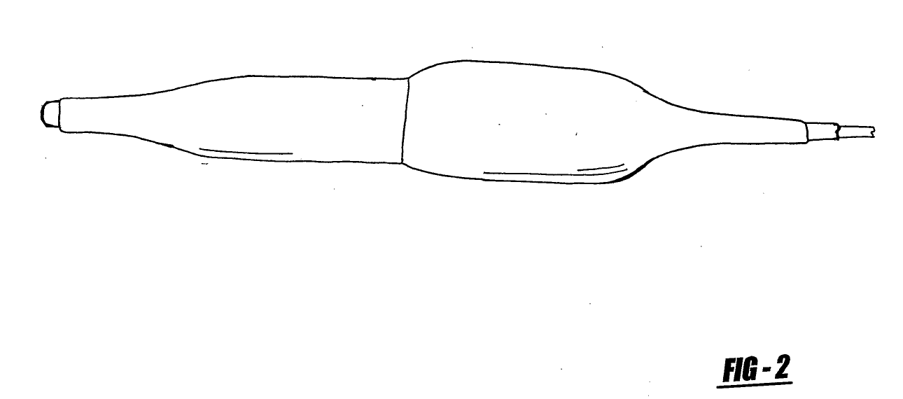

An example complex medical balloon is shown in an inflated condition in FIG. 2. The balloon is inflated at a specific pressure, and at that pressure it has a larger segment and a smaller segment. The larger segment is made of materials having a compliance curve that rises above the compliance curve of the smaller segment. The two segments may also be described as having relatively “higher compliance” and “lower compliance.”

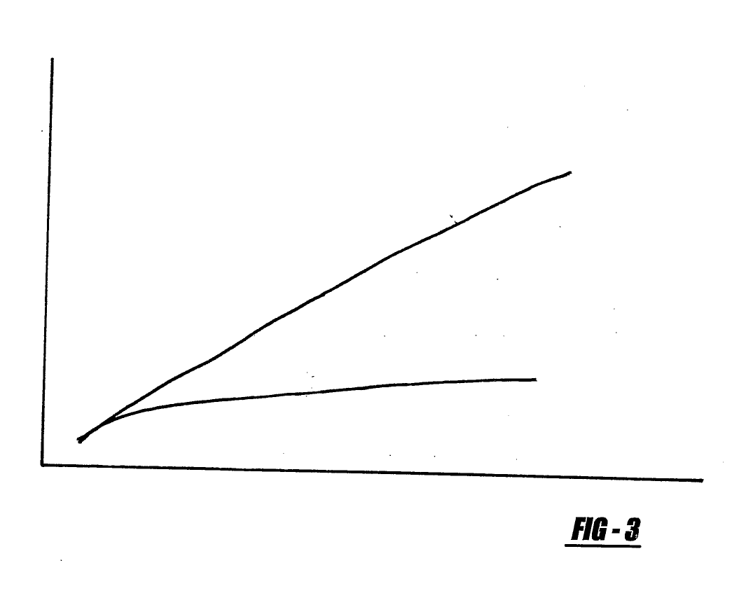

Complex medical balloons of the present invention have compound compliance curves, and a diagrammatic representation is shown in FIG. 3. A compliance curve is a graph of the size of a balloon segment, when the balloon is inflated to a variety of pressures. A compound compliance curve occurs when different portions of the same balloon exhibit different sizes at the same inflation pressure. Accordingly, the lower compliance curve corresponds to the smaller or lower compliance segment of the complex medical balloon, and the higher compliance curve corresponds to the larger or higher compliance segment.

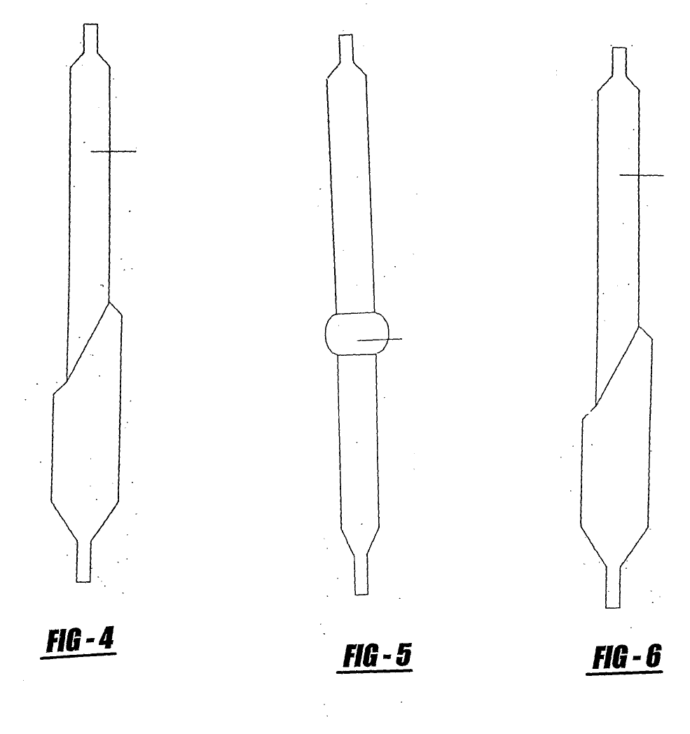

FIG. 4 shows a complex medical balloon having segments of differing compliance. Multiple diameters and/or multiple material balloon parisons are possible. Two parisons of different dimensions and/or materials may be fused together using techniques that insure that both parisons melt. The resulting combined parison is stretched and blow-molded using multiple or single diameter molds. Balloons that form shapes or multiple sizes at varying pressures are produced.

The relatively non-distensible section can be made of any existing or suitable polymers used in medical dilatation balloons. Polyamides of the 12 type are examples. The more distensible section is made with a softer polymer such as lower durometer polyether-block-amide. As an example the less distending section may be made of polyamide-12 homopolymer (VESTAMID manufactured by Degussa, Germany). The more distensible section may be made of a polyamide 12 based polyether-block-amide of lower durometer (PEBAX manufactured by Atofina, France).

FIG. 5 shows a complex medical balloon with a center segment having a different compliance curve. During balloon blowing, heat is applied at specific locations. When heat is applied to specific locations for the balloon/parison, before full expansion, the wall location of high temperature is thinner. The thinner wall portions of the balloon expand a relatively greater amount than the thicker locations.

Any of the existing or suitable polymers used in medical balloons used for dilatation. Polyamide of the 12 types are examples.

FIG. 6 shows a complex medical balloon having one balloon, with a portion of the balloon having a sleeve surrounding and bonded to it. For example, a bi-axially oriented sleeve that is relatively inelastic covers a section of the balloon. When the balloon is expanded, the inelastic section limits the expansion of that section. The sleeve can be bonded to the balloon with adhesives at room temperature or during the balloon-blowing process.

One method uses a coextruded sleeve and balloon material. The parison for the sleeve consists of an outside layer of relatively inelastic polymer and an inside layer of a lower melting point polymer. The balloon parison is comprised of an outside layer of a lower melting point polymer and an inside layer of a polymer with elevated compliance. The balloon and sleeve are bonded by applying heat and pressure. The temperature used would be less than the melt point of the parisons inside the sleeve's outside layer and the sleeve's inside layer.

The sleeve can be made of any existing or suitable polymer used in medical dilatation balloons. Polyamides of the 12 type are examples. The balloon body that the sleeve is attached to is made from a softer more compliant polymer than the sleeve. Polyetherurethanes are examples. An example is a bi-axially oriented balloon body made from co-extruded parison with an inner layer of Pellethane and an outer layer made from PEBAX manufactured by Dow Chemical, U.S.A. The sleeve is made from bi-axially oriented tubing with an inner layer of PEBAX and an outer layer of Vestamid. The sleeve is attached to the balloon body by applying a temperature above the melting point of the PEBAX and below the melting point of Vestamid or Pellethane. The temperature is applied while the balloon and sleeved are constrained by a mold.

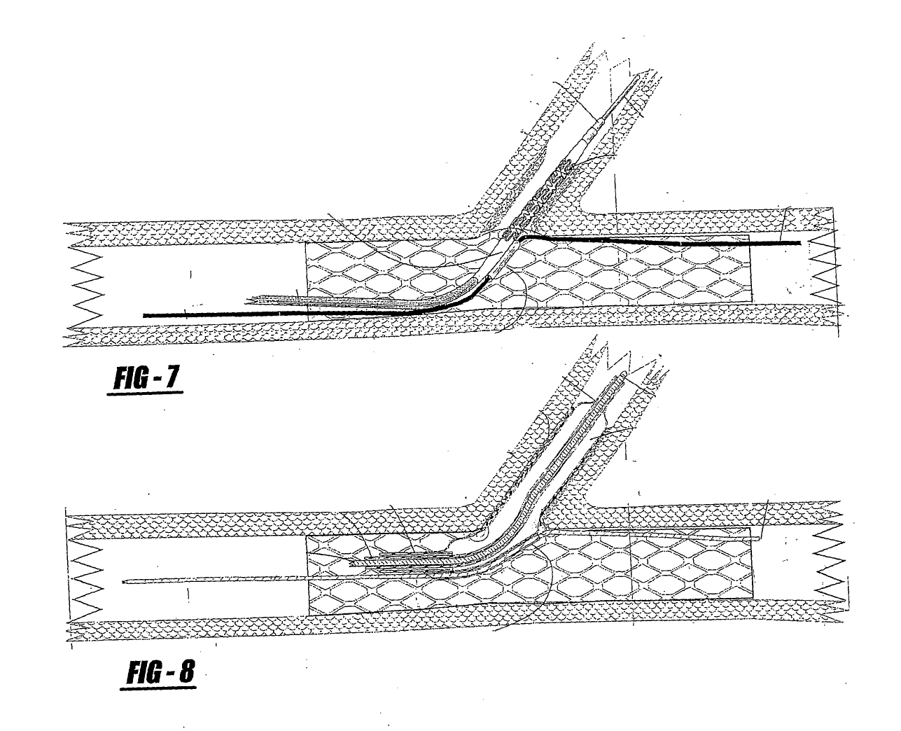

In FIGS. 7 and 8, a method of stenting bifurcated vessels is to deploy two cylindrical stents is depicted, one in the side branch artery and one in the main artery. The stent may cover all of the vessels including the area of junction. Continuous coverage is thought to be desirable when drug-eluting stents are used. The end of the branched vessel is tapered at the junction with the main vessel. Therefore the use of two cylindrical stents will not adequately conform to the vessel wall at the junction. Balloons of this invention allow the interventional cardiologist to flare the end of the stent in the branched vessel while deploying the side branched stent. This flared end allows the stent to conform to vessel junction and provide continuous coverage. The amount of flaring can be controlled by the amount of pressure applied to the balloon.

In prior balloons, a tubular polymer parison that is bi-axially oriented into a mold of differing diameters produces a balloon with differing sizes, but the difference in the two sizes decreases as the balloon is pressurized. At a certain pressure, the sizes will approximate or equal each other.

This type of balloon would be of limited use to flare a stent end. The difference in diameters of the balloons of this invention increase as the pressure increases. This feature allows the body of the side branch stent to be fully deployed without excessive distention while the amount of flaring of the stent end is controlled by the amount of pressure applied.

When a complex medical balloon is used in a balloon catheter or stent delivery system, the remaining portions may be made using various methods, including extruding polymer tubes, injection-molding the proximal hub, and extruding a balloon parison and then blowing the parison into a balloon having the desired properties. It is also possible to affix polymer components to each other by heat-sealing, or by using an adhesive such as a UV-cured adhesive.

Example:

A complex medical balloon was constructed of a main balloon component, and a sleeve component which surrounded and was bonded to a portion of the balloon.

The main balloon component was made of a coextruded parison, in which the outer layer was anhydride-modified low-density polyethylene (LDPE), and the inner layer was polyurethane (PU). The sleeve component was also made of a coextruded parison, in which the outer layer was a nylon homopolymer, and the inner layer was anhydride-modified low-density polyethylene (LDPE).

In both components, the wall thickness of the LDPE was much thinner than that of the other layer, on the order of 10%. Also, it should be mentioned that the LDPE has a melting temperature lower than either of the other materials.

Both the main balloon component and the sleeve component were bi-axially oriented using known techniques. In other words, the sleeve component was bi-axially oriented and formed into a balloon type of shape, and then a desired length of the sleeve was cut from it. The sleeve was positioned over one end of the main balloon component, and the assembly was placed in a mold and pressurized. The mold was heated, before or after pressurization, above the melt pointing of the LDPE (and below the melting points of the other polymers). This temperature was on the order of 110 degrees Celsius. The two balloon components were thus bonded together. The mold is then cooled to room temperature. The resulting balloon exhibited a compound compliance curve similar to FIG. 3, and an average burst pressure of as high as 23 atmospheres.

Of course, some alternative constructions and methods are possible, including making one of the component of a single layer material, or bonding the parisons together before bi-axially orienting them into a balloon.

Other Treatments:

In some treatments, medical stents placed inside branching vessels, called bifurcations, would benefit from a flared end. The stent will conform better to the area where the two vessels join. Some balloons according to the present invention provide two diameters when pressurized. The difference in the diameters increases with increasing pressure.

One end of the balloon, where the body of the stent is mounted, is relatively noncompliant. This allows for effective deployment of a stent in the side branch vessel. The other end of the balloon is more compliant. This section of the balloon expands to a larger diameter under pressure and flares the end of the stent in the area where the two vessels join.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A complex medical balloon for medically treating a patient, comprising:
a balloon defining an interior and having a first and second portion;
the first and second portions exhibiting different compliance curves, wherein the compliance curves for the first portion and the second portion diverge at greater pressures.

2. A complex medical balloon for medically treating a patient, comprising:
a balloon defining an interior and having a first and second portion being made of different polymers;
the first and second portions exhibiting different compliance curves.
